(19) United States
(12) Patent Application Publication
(10) Pub. No.: US 2026/0352949 A1
(43) Pub. Date: Nov. 5, 2026

(54) FOCUS CONTROL DEVICE, FOCUS CONTROL METHOD, AND COMPUTER-READABLE STORAGE MEDIUM FOR FOCUS CONTROL OF AN ENDOSCOPE

(71) Applicant: OLYMPUS MEDICAL SYSTEMS CORP., Tokyo (JP)

(72) Inventor: Tomohiro HOSHINO, Tokyo (JP)

(73) Assignee: OLYMPUS MEDICAL SYSTEMS CORP., Tokyo (JP)

(21) Appl. No.: 19/711,287

(22) Filed: Jun. 17, 2026

Related U.S. Application Data
(63) Continuation of application No. PCT/JP2023/045999, filed on Dec. 21, 2023.

(51) Int. Cl. H04N 23/67; H04N 23/50

(52) U.S. Cl. CPC H04N 23/67; H04N 23/555

(57) ABSTRACT

A focus control device for an endoscope includes a connection interface and processing circuitry. The processing circuitry is configured to receive, via the connection interface, images obtained by an imaging device disposed at a distal end portion of the endoscope, detect a change in the images, and determine an observation target site from the images based on a correspondence between the detected change in the images and an operation to change an imaging range of the imaging device. The processing circuitry is further configured to determine the observation target site as a focusing target and perform focus control of the imaging device based on the focusing target.

### CROSS REFERENCE TO RELATED APPLICATION

This application is a continuation of International Application No. PCT/JP2023/045999, filed on Dec. 21, 2023, the entire contents of which are incorporated herein by reference.

### FIELD

The present disclosure relates to an endoscope focus control device, a focus control method, and a focus control program that facilitate focusing.

### BACKGROUND

An endoscope is a device that is inserted into a body or the like to enable observation of an affected part or the like not visible from outside. The endoscope includes an elongated insertion portion having flexibility that is inserted into a body cavity of a patient, for example. The endoscope picks up an image of each part in the body by an image pickup device provided at a distal end of the insertion portion. The image (endoscopic image) obtained by the endoscope is supplied to a video processor for image processing. The endoscopic image from the video processor is displayed on a display screen of a monitor.

The image to be photographed is obtained using an optical system in a deep focus state, but some endoscopes also have an autofocus control function to acquire an image of a target in focus according to a distance from the target. Note that regarding an autofocus control technology applied to a digital camera, there is a technology disclosed in Japanese Patent Application Laid-Open Publication No. 2005-31200 (hereinafter referred to as Patent Document 1). In the technology of Patent Document 1, when an object is lost during pattern-driven AF control (normal control state), the pattern-driven AF control (extended control state) is continued until at least a predetermined time has elapsed.

### SUMMARY

According to aspects of the present disclosure, a focus control device for an endoscope is provided, which includes a connection interface and processing circuitry. The processing circuitry is configured to receive, via the connection interface, images obtained by an imaging device disposed at a distal end portion of the endoscope. The processing circuitry is further configured to detect a change in the images. The processing circuitry is further configured to determine an observation target site from the images based on a correspondence between the detected change in the images and an operation to change an imaging range of the imaging device. The processing circuitry is further configured to determine the observation target site as a focusing target. The processing circuitry is further configured to perform focus control of the imaging device based on the focusing target.

According to aspects of the present disclosure, further provided is a focus control method implemented by processing circuitry communicably connected to an endoscope. The focus control method includes receiving images obtained by an imaging device disposed at a distal end portion of the endoscope. The focus control method further includes detecting a change in the images. The focus control method further includes determining an observation target site from the images based on a correspondence between the detected change in the images and an operation to change an imaging range of the imaging device. The focus control method further includes determining the observation target site as a focusing target. The focus control method further includes performing focus control of the imaging device based on the focusing target.

According to aspects of the present disclosure, further provided is a non-transitory computer-readable storage medium storing instructions executable by processing circuitry communicably connected to an endoscope. The instructions are configured to, when executed by the processing circuitry, cause the processing circuitry to receive images obtained by an imaging device disposed at a distal end portion of the endoscope. The instructions are further configured to, when executed by the processing circuitry, cause the processing circuitry to detect a change in the images. The instructions are further configured to, when executed by the processing circuitry, cause the processing circuitry to determine an observation target site from the images based on a correspondence between the detected change in the images and an operation to change an imaging range of the imaging device. The instructions are further configured to, when executed by the processing circuitry, cause the processing circuitry to determine the observation target site as a focusing target. The instructions are further configured to, when executed by the processing circuitry, cause the processing circuitry to perform focus control of the imaging device based on the focusing target.

### BRIEF DESCRIPTION OF DRAWINGS

FIG. 1 is a block diagram showing an endoscope focus control device according to an embodiment of the present disclosure.

FIG. 2 is a flowchart for explaining an operation of the embodiment.

FIG. 3 is an explanatory illustration showing a state of endoscopy.

FIG. 4 is an explanatory illustration showing the state of the endoscopy.

FIG. 5 is a flowchart showing an example of a specific operation flow of step S4 in FIG. 2.

FIG. 6 is an explanatory illustration for explaining an image pickup range by bold broken lines.

FIG. 7 is an explanatory illustration for explaining examples where a plurality of target patterns exist and where an observation target part does not include a specific pattern.

### DESCRIPTION OF EMBODIMENTS

With an endoscope, it is relatively difficult to capture a desired observation target part within an image pickup range. Therefore, even when utilizing the technology of Patent Document 1, it is not always possible to reliably focus on the desired observation target part.

In the present specification, there are described an endoscope focus control device, a focus control method, and a focus control program that enable reliable focusing on a desired observation target part by performing autofocus control utilizing, for example, an operation to follow and observe the observation target part.

According to the present disclosure, reliable focusing on a desired observation target part can be performed by determining an autofocus target utilizing, for example, an operation to follow and observe the observation target part according to the observation target part and controlling the autofocus target.

Hereinafter, an embodiment of the present disclosure will be described in detail with reference to the drawings.

### Embodiment

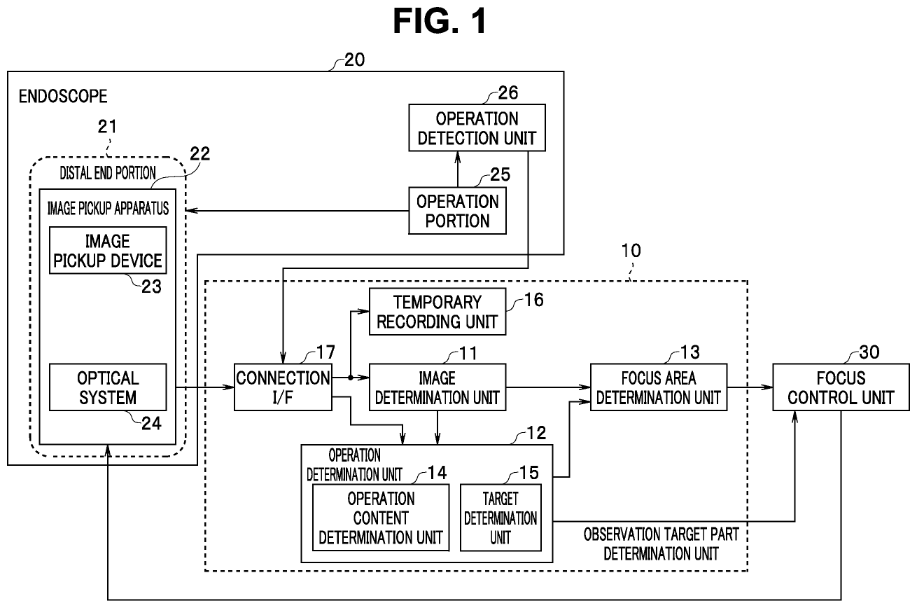

FIG. 1 is a block diagram showing an endoscope focus control device according to an embodiment of the present disclosure. The present embodiment enables reliable focusing on a desired observation target part by determining an operation applied to an endoscope for capturing the observation target part within an image pickup range, for example at its center, to pick up an image, and performing autofocus control based on the determination result.

The endoscope has to pick up not only an image of a target with distinct features, such as a face, but also an image of a target with indistinct features. Therefore, even when employing an autofocus technology used in a camera, a smartphone, or the like, which focuses on a specific target such as a face, it is not always possible to reliably focus on a desired observation target part. In addition, in a case of image pickup by the endoscope, organs or the like in a body cannot be moved to achieve an appropriate composition. Consequently, not only is it impossible to pick up an image of the observation target part with an optimal composition, but also it may be difficult to easily position the observation target part within the image pickup range.

As features of the endoscope, an operation portion and an image pickup unit (image pickup apparatus 22 described later), which is often provided near a distal end, are distant from each other, so a movement of a hand operating the operation portion is not directly transmitted to a distal end of an insertion portion. In addition, there is little feedback regarding a movement of the distal end in response to the operation, making an intuitive operation difficult. Furthermore, the insertion portion moves while being in contact with structures in the body, but a state where the insertion portion is in contact with the structures is not picked up in the image pickup range of the image pickup unit disposed at the distal end of the insertion portion. Consequently, even when viewing the picked up image, it is impossible to identify factors hindering the movement of the insertion portion. This makes movement of the insertion portion itself difficult, so framing during image pickup through the endoscope operation is subjected to significant constraints. In other words, an operator cannot capture a desired target as intended, making focusing difficult. Consequently, problems such as requiring time for essential observation and examination may arise. While such a situation is particularly noticeable with a flexible endoscope including a flexible portion, it can also occur with an endoscope (referred to as a rigid endoscope) that includes fewer flexible portions.

Therefore, the present embodiment enables focusing on a desired observation target part, reflecting an intent of an operator. Hereinafter, the observation target part may also be referred to as the observation target site.

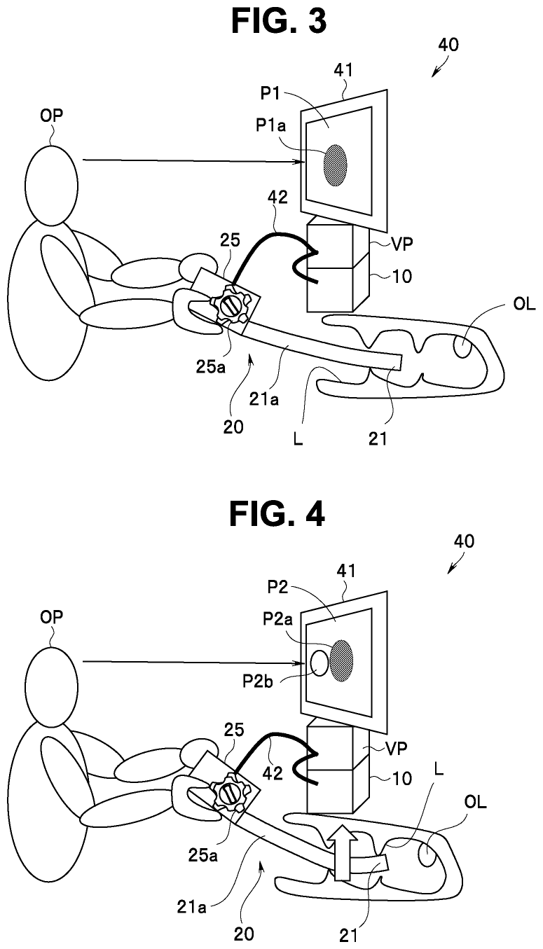

In FIG. 1, the endoscope focus control device includes an observation target part determination unit 10 configured to receive an endoscopic image from an endoscope 20. The endoscope focus control device may further include a focus control unit 30. In other words, the observation target part determination unit 10 and the focus control unit 30 may form the endoscope focus control device (hereinafter also referred to as the focus control device for the endoscope 20). The endoscope focus control device (including the observation target part determination unit 10 and the focus control unit 30) may be implemented by processing circuitry including one or more processors and memories. Note that although a specific configuration for displaying the endoscopic image is not shown in FIG. 1, by supplying the endoscopic image, whose focus is controlled by the endoscope focus control device of the present embodiment, to a video processor (video processor VP shown in FIG. 3) to perform image processing, it is possible to display the endoscopic image focused on the observation target part on a monitor.

The endoscope 20 includes an elongated insertion portion having flexibility, to be inserted into a body cavity of a subject. A distal end portion 21 is provided at a distal end of the insertion portion. An operation portion 25 to which various operation devices are disposed is provided on a proximal end side of the insertion portion. The insertion portion includes a bendable bending portion (not shown) configured by a plurality of bending pieces. By operating the operation portion 25 to drive the bending portion, an orientation of the distal end portion 21 can be directed in up, down, left, and right directions. The operation portion 25 and the bending portion are connected by a plurality of wires and the like, such that operations of pulling or loosening the wires are transmitted to the distal end. However, in recent years, types using electrical connection to operate an actuator at the distal end via switches have also been proposed. When the operation portion 25 is, for example, a lever or a knob, a degree of bending can be changed depending on an amount of rotation or an amount of operation thereof. The amount of rotation or the amount of operation may be electrically detected by a sensor or the like, but an amount of movement corresponding to the amount of rotation or the amount of operation may be detected from a change in images as an image pickup result obtained by the image pickup unit (image pickup apparatus 22) at the distal end. Since the operator performs the operation while viewing the images, image changes (changes over time) occur according to the intent of the operator.

The image pickup apparatus (hereinafter also referred to as the imaging device) 22 is provided at the distal end portion 21 of the distal end of the insertion portion. The image pickup apparatus 22 includes an image pickup device 23, and an optical system 24 that guides an object optical image to an image pickup surface of the image pickup device. The image pickup device 23 is configured by a CCD sensor, a CMOS sensor, or the like, and photoelectrically converts the object optical image from the optical system 24 to acquire a picked-up image (image pickup signal) of an object. The optical system 24 includes lenses, apertures, and the like which are not shown, for zooming and focusing, and also includes a zooming (variable magnification) mechanism and a focus and aperture mechanism, which are not shown, for driving these lenses. Image information of the picked-up images (endoscopic images) sequentially obtained in time series by the image pickup apparatus 22 is supplied to the observation target part determination unit 10.

During an examination or a treatment using the endoscope 20, an operator such as a physician operates the endoscope 20 to insert the insertion portion of the endoscope 20 into a lumen of a human body. The physician operates a bending knob 25a or the like provided on the operation portion 25, to bend the bending portion or advance/retract the insertion portion, thereby moving the distal end portion 21 of the insertion portion to the observation target part. During endoscopy, it is necessary to insert the insertion portion according to a state of the lumen, which is narrow or curved, so it is relatively difficult to move the distal end portion 21 to a desired position and orient the distal end portion 21 in a desired orientation. Therefore, in order to obtain a high quality observation image focused on the observation target part the physician desires, cumbersome operations may be required.

Therefore, the observation target part determination unit 10 determines the observation target part by determining features of the images obtained by the image pickup apparatus 22 and changes over time of the features of the images, as well as determining operations by the operator on the distal end portion 21 at which the image pickup apparatus 22 is disposed. In the present embodiment, the image pickup apparatus 22 is automatically focus-controlled using the determination result of the observation target part determination Unit 10.

The observation target part determination unit 10 includes an image determination unit 11, an operation determination unit 12, a focus area determination unit 13, and a temporary recording unit 16. The observation target part determination unit 10 may include a connection interface (hereinafter also referred to as a connection I/F) 17 configured to receive image data from the image pickup apparatus 22 and operation information from the operation detection unit 26. The connection I/F 17 may distribute the received image data and operation information to respective functional units including the image determination unit 11, the temporary recording unit 16, and the operation determination unit 12. The connection I/F 17 may be implemented as a wired and/or wireless interface, and may include, for instance, a communication interface, an input port, and/or a data acquisition interface. Each unit of the observation target part determination unit 10 and the focus control unit 30 may be implemented by processing circuitry, which includes one or more processors, such as a CPU (central processing unit), an FPGA (field programmable gate array), or the like, and further includes one or more memories (non-transitory computer-readable storage media). Each unit of the observation target part determination unit 10 and the focus control unit 30 may be controlled by operating according to a program stored in a memory (not shown), or may achieve at least one of functions using a hardware electronic circuit.

The image from the endoscope 20 is supplied to the temporary recording unit 16 and the image determination unit 11 of the observation target part determination unit 10. The temporary recording unit 16 is configured by a predetermined memory medium, and temporarily records the endoscopic image. The image determination unit 11 performs various image analysis processing on the endoscopic image and sequentially determines features of each portion in the image, such as various patterns in the endoscopic image. The image determination unit 11 supplies the determination result to the operation determination unit 12 and the focus area determination unit 13.

The operation determination unit 12 includes an operation content determination unit 14 and a target determination unit 15. The operation content determination unit 14 detects a time-series change in the features of each portion (for example, a pattern such as lesion part) in the endoscopic images, based on the determination result of the image determination unit 11, and determines an operation content by the operator for causing the image pickup range of the image pickup apparatus 22 to change. Specifically, the operation content determination unit 14 determines the operation content by the operator for moving or changing the orientation of the distal end portion 21 of the endoscope 20 to cause the image pickup range of the image pickup apparatus 22 to change. Note that the operator moves or changes the orientation of the distal end portion 21 by moving the entire endoscope 20 or by operating the lever or the switches (not shown) of the operation portion 25.

For example, when the endoscope is inserted into the lumen, the distal end portion 21 of the endoscope 20 moves, and the endoscopic image changes such that the image pattern at the center of the image moves toward the periphery of the image. In addition, the endoscopic image during withdrawal of the endoscope changes such that the image pattern at the periphery of the image moves to the center of the image. Furthermore, with the flexible endoscope, an operation to bend the distal end may be performed in addition to insertion and withdrawal operations. During the bending operation, the image pattern in the endoscopic image changes its position over time in up, down, left, and right directions on the XY two-dimensional coordinates of the image.

The operation content determination unit 14 obtains, based on the determination result of the image determination unit 11, changes in each portion in the images sequentially obtained in time series by comparing preceding and following images or analyzing motion vectors, and based on the obtained result, determines the operation content by an operator such as a surgeon, including a type of the operation and an amount of the operation, such as inserting or withdrawing the insertion portion (distal end portion 21), or changing the orientation of the distal end portion 21 by the bending operation. Note that the operation content determination unit 14 may determine the operation content by AI (artificial intelligence) processing using an inference model for determining the operation content.

Note that the endoscopic image changes not only due to the movement of the insertion portion (distal end portion 21) but also due to movement of the organs or the like, which is an object. However, a manner of the change in the image due to the movement of the insertion portion differs from a manner of the change in the image due to the movement of the organ or the like. Therefore, the operation content determination unit 14 can detect the changes in the image due to the movement of the insertion portion through image analysis.

The target determination unit 15 determines the observation target part desired for observation by the surgeon based on the determination result of the image determination unit 11 and the determination result, obtained by the operation content determination unit 14, of the operation content by the surgeon. For example, depending on the operation content by the surgeon, the target determination unit 15 may determine the part picked up at the center of the image as the observation target part. In addition, for example, when the operation content by the surgeon indicates a specific direction, the target determination unit 15 may determine the part existing in the specific direction as the observation target part. Furthermore, for example, when a lesion part is picked up in the image, the target determination unit 15 may determine the lesion part as the observation target part.

Due to the constraints on the framing described above, the observation target part desired for observation by the surgeon may not always be picked up at the center of the image. However, since the operator attempts to adjust the operation so as to position the observation target part at the center of the image, the target determination unit 15 may identify the observation target part by determining such an operation based on the determination result of the operation content determination unit 14.

The example is described where the operation determination unit 12 determines the operation content and the observation target part based on the endoscopic image, but the operation determination unit 12 may determine the operation content and the observation target part based on other information. In the present embodiment, the operation detection unit 26 acquires operation information based on the operation on the operation portion 25. The operation determination unit 12 may determine the operation content by the surgeon and the observation target part using the operation information from the operation detection unit 26.

The operation detection unit 26 may acquire the operation information using various sensors. For example, the operation detection unit 26 may detect an amount of operation of the bending knob 25a provided on the operation portion 25 to acquire the operation information. In addition, for example, the operation detection unit 26 may detect an insertion length of the insertion portion to acquire the operation information. Furthermore, the operation detection unit 26 may utilize a well-known endoscope insertion shape observation device for detecting an insertion shape of the insertion portion by transmission and reception coils provided at the distal end portion 21 and an external fixed position to detect an operation by the operator that moves the distal end portion 21 and changes the orientation of the distal end portion 21, thereby acquiring the operation information.

Furthermore, the operation detection unit 26 may acquire the operation content by the operator that causes the image pickup range of the image pickup apparatus 22 to change by voice recognition processing on a voice acquired using a microphone that acquires ambient sound. When the operator utters a voice such as “more to the right” while moving the distal end portion 21, for example, the operation detection unit 26 can acquire the operation content based on the content of the uttered voice.

The operation determination unit 12 may constantly determine the operation content and the observation target part during image pickup by the endoscope, or may determine the operation content and the observation target part only during a specific period. For example, the operation determination unit 12 may perform determination when transitioning from a state where screening is being performed by the endoscope to a state of observing the specific part (observation target part). In addition, the operation determination unit 12 may perform determination when approaching the observation target part and zooming in on the part for observing the specific part (observation target part). The operation determination unit 12 may decide whether to perform determination of the operation content and the observation target part based on the determination result of the image determination unit 11 or the operation information of the operation detection unit 26. For example, when the operator operates the operation portion 25, starting of the determination may be instructed. Note that when the determination is not performed, the operation determination unit 12 may output a signal indicating that focus control is not necessary to the focus control unit 30. In other words, as described later, when the determination result of the operation content indicating that the state of screening by the endoscope has been transitioned to the state of observing the above observation target part is obtained by the operation determination unit 12, the focus control on a focus area may be performed.

The focus area determination unit 13 is supplied with the determination result of the image determination unit 11 and an estimation result of the observation target part from the operation determination unit 12, to set the focus area, which is a target region for focusing in the endoscopic image. The focus area indicates a region where focusing is performed in the image pickup range of the image pickup apparatus 22. The focus area determination unit 13 determines a region in the image pickup range where the observation target part obtained by the target determination unit 15 exists, as the focus area to be set. Information on the focus area determined by the focus area determination unit 13 is supplied to the focus control unit 30.

The focus control unit 30 controls the image pickup apparatus 22 such that the focus area set by the focus area determination unit 13 is in focus. The focus control unit 30 controls a focus mechanism of the optical system 24 to bring the focus area in focus. Note that during a period indicated by the operation determination unit 12 as requiring no focus control, the focus control unit 30 does not perform focus control. Note that the focus control unit 30 may prioritize focusing on a specified focus area over other areas, or may add a predetermined weight to the focusing on the focus area.

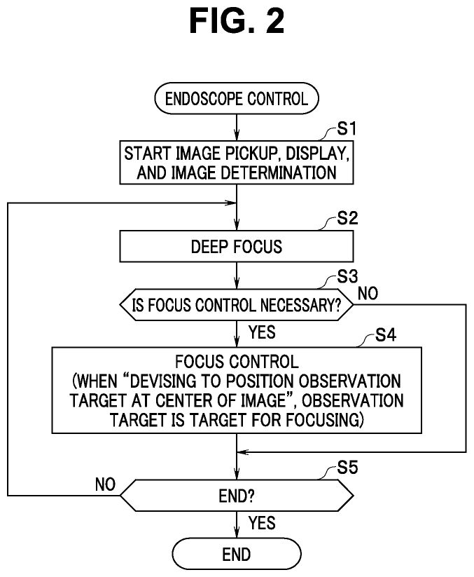

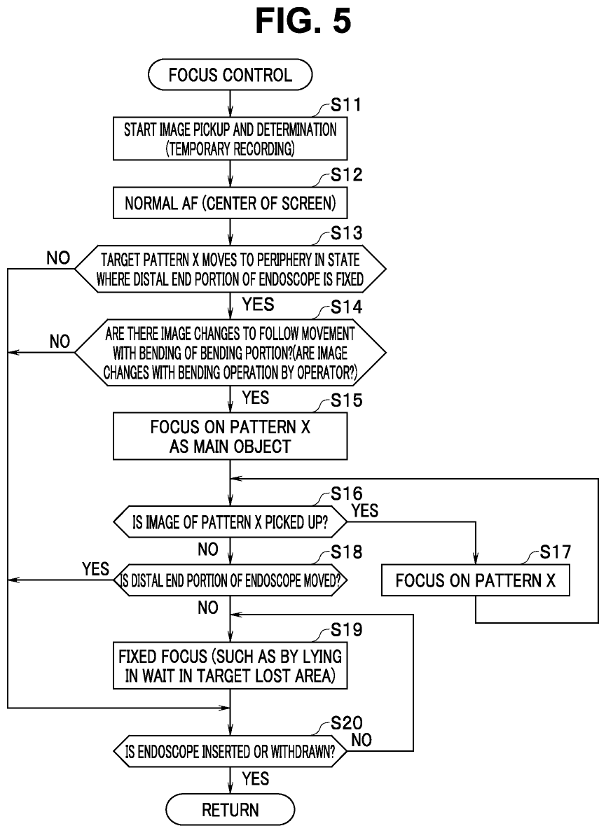

Next, the operation of the embodiment thus configured will be described with reference to FIG. 2 to FIG. 7. FIG. 2 is a flowchart for explaining the operation of the embodiment, and FIG. 3 and FIG. 4 are explanatory illustrations showing a state of the endoscopy. FIG. 5 is a flowchart showing an example of a specific operation flow of step S4 in FIG. 2.

In the following description, a procedure of image pickup of a relatively wide range to search for the lesion part, or the like is referred to as “screening”, and a procedure of image pickup of a specific part in detail for confirmation, for example, by moving close to the specific part to pick up an image or zooming in, is referred to as “observation”. The following description will be given assuming that, for example, during screening, deep focus or autofocus control at the center of the image pickup range is performed, and during observation, focus control is performed by the focus control unit 30. However, it is not limited thereto.

FIG. 3 shows a state of an examination by an endoscope system 40 including the observation target part determination unit 10. An operator OP inserts an insertion portion 21a of the endoscope 20 into a lumen L. The endoscope 20 is connected to a video processor VP and the observation target part determination unit 10 via a cable 42. An endoscopic image from the image pickup apparatus 22 (see FIG. 1) disposed at the distal end portion 21 of the endoscope 20 is supplied to the video processor VP and the observation target part determination unit 10. The video processor VP performs predetermined image signal processing on the inputted endoscopic image, and supplies the endoscopic image to a monitor 41 to cause the endoscopic image to be displayed.

In S1 of FIG. 2, image pickup, display, and image determination are started. Specifically, the image pickup apparatus 22 disposed at the distal end portion 21 of the insertion portion 21a starts the image pickup, the endoscopic image is supplied to the video processor VP, and the image of the lumen L is displayed on the monitor 41. At the time of starting the screening, image pickup in deep focus is performed, for example (S2).

In the example of FIG. 3, an image of the lumen L is picked up by the image pickup apparatus 22 disposed at the distal end portion 21 of the insertion portion 21a inserted into the lumen L, and an image P1 inside the lumen L is displayed on a display screen of the monitor 41. Note that the image P1a shows a dark portion in a deep direction of the lumen L. The operator OP performs screening of the lumen L by moving the distal end portion 21 while viewing the endoscopic image displayed on the monitor 41, as shown by the arrow representing a line of sight.

The operation determination unit 12 determines in S3 whether focus control is necessary. For example, the operation such as observing a specific part (observation target part) from the state of screening using the endoscope or picking up an image of the observation target part from a close position and zooming in on the observation target part is performed. In this case, the operation determination unit 12 determines, for example, based on the determination result of the image determination unit 11, that focus control is necessary. Then, focus control is performed in S4. When determining that focus control is not necessary (NO in S3), the operation determination unit 12 proceeds to S5 to determine whether the endoscopy is ended.

Note that the operation determination unit 12 may determine whether a desired part to be observed exists and whether focus control is necessary by considering the time to pick up an image of a specific part. For example, the operation determination unit 12 may determine that the observation target part exists in the image pickup range and focus control is necessary when image pickup of the same part continues for one second or longer.

FIG. 4 shows an example during observation. The operator OP inserts the insertion portion 21a into a deep portion side of the lumen L and is performing screening. Here, the operator OP discovers a part that may be a lesion part OL in the deep portion of the lumen L, approaches the part from the screening state, for example, and zooms in and attempts to magnify the part (observation target part) for observation. In FIG. 4, an endoscopic image P2 immediately before transitioning to observation is displayed on the display screen of the monitor 41. At the center of the endoscopic image P2, an image P2a of a dark portion of the deep portion of the lumen L is displayed, and an image P2b of the lesion part OL is displayed at an edge of the image.

The operator OP advances the distal end portion 21 further into the deeper portion side of the lumen L, and operates the bending knob 25a to orient the distal end portion 21 in a direction indicated by the arrow in FIG. 4, aiming to position the lesion part OL at the center of the image pickup range. However, as shown in the example of FIG. 4, the insertion portion 21a may come into contact with a protrusion, a fold, or the like on a lumen wall of the lumen L, making it difficult to orient the distal end portion 21 in a direction of the lesion part OL. Even in this case, in the present embodiment, it is possible to pick up and display an image of the lesion part OL on the monitor 41 in focus. In some cases, an enlarged image of the lesion part OL can be displayed at the center of the image.

In other words, the operation determination unit 12 determines that focus control is necessary using the determination result of the image determination unit 11 and the operation information of the operation detection unit 26 (YES in S3). The operation determination unit 12 determines the operation content in S4. For example, the operation content determination unit 14 obtains the operation content indicating that the operator OP adjusts the operation so as to position the target at the center of the image (the center of the image pickup range). The target determination unit 15 determines the target as the observation target part. In this case, the focus area determination unit 13 sets an area where the object is likely to exist as the focus area. Thus, the focus control unit 30 controls the optical system 24 of the image pickup apparatus 22 such that the focus area is in focus. Consequently, the picked-up image of the lesion part OL is in focus and the image P2b on the monitor 41 becomes an easily visible image.

If the endoscopy is not ended (NO in S5), the process returns to S2, and screening is continued. If the endoscopy is determined to be ended (YES in S5), the process ends.

Next, an example of focus control in step S4 will be described in detail with reference to FIG. 5 to FIG. 7.

Note that in the following description, an example of performing autofocus control by determining an operation that moves the image pickup range following the movement of the observation target part to the periphery of the image pickup range so as to position the observation target part at the center of the image pickup range will be described. However, transition to autofocus control is not limited to such a case. For example, autofocus control may be performed based on an operation to position the observation target part located from the periphery of the image pickup range to the center of the image pickup range. Alternatively, autofocus control may be performed based on a zoom operation or the like for observing the observation target part located at the center of the image pickup range.

In S11 of FIG. 5, image pickup by the endoscope 20 and determination by the observation target part determination unit 10 are started. The endoscopic image acquired by the endoscope 20 is temporarily recorded in the temporary recording unit 16. Normal autofocus (AF) control is performed on the image pickup apparatus 22 to focus on the center of the image pickup range (S12). The operation content determination unit 14 determines whether a target pattern X moves to the periphery of the image pickup range in a state where the distal end portion 21 is fixed, that is, in a no-operation state where the operator OP does not apply any operation to the endoscope, in S13.

As described above, the endoscopic image changes due to movement of the distal end portion 21 and movement of the organ, which is the object. In the present embodiment, the focus area is decided by determining the operation by the operator OP to follow the movement of the observation target part. In S13, the operation determination unit 12 determines whether the image changes due to the movement of the organ, not due to the operation by the operator OP. If the image changes are due to the operation by the operator OP (NO in S13), the process transitions to S20. If the change in the target pattern X is due to the movement of the organ, the operation determination unit 12 determines the operation content in S14.

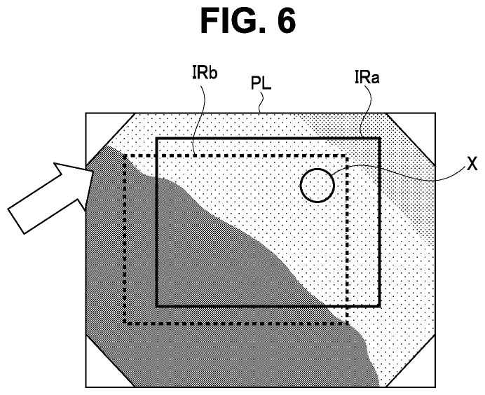

FIG. 6 is an explanatory illustration for explaining the image pickup range in this case by bold broken lines. The target pattern X, such as the lesion part, exists in the lumen wall PL, which is the object, and FIG. 6 shows a state where the target pattern X moves to the upper right edge of the image pickup range IRb due to the movement of the organ, or the like. In order to observe the target pattern X, the operator OP performs various operations to position the target pattern X at the center of the image pickup range. The operation content determination unit 14 determines the operation content of such an operation by the operator OP.

For example, the operator OP operates the operation portion 25 to bend the bending portion of the insertion portion 21a, thereby following the movement of the target pattern X in the picked-up image to cause the image pickup range to change. The operation content determination unit 14 determines that, based on the image changes in this case, the operator OP performs the operation of the operation content that operates the bending knob 25a to bend the insertion portion 21a and follow the movement of the target pattern X to cause the image pickup range to change.

FIG. 6 shows that the image pickup range changes in a direction indicated by the arrow and changes to an image pickup range IRa indicated by the bold solid lines. If there are image changes with such a change to the image pickup range IRa (YES in S14), the target determination unit 15 determines that the target pattern X is a main object (observation target part) based on the determination result of the operation content determination unit 14 and an output of the image determination unit 11, and the focus area determination unit 13 sets a region in the image pickup range where the target pattern X exists as the focus area. The focus control unit 30 controls the optical system 24 of the image pickup apparatus 22 to focus on the set focus area (S15). Note that the operation determination unit 12 proceeds to the process to S20 if there are not image changes as described above (NO in S14).

Specifically, in the present embodiment, by determining the operation by the operator OP to follow the target pattern X, the region in the image pickup range corresponding to the target pattern X is focused on as the focus area. In other words, if an image feature determination unit that determines features of the image obtained by the image pickup unit (image pickup apparatus 22) at the distal end portion of the endoscope is provided, in the picked-up image, a position change (move upward, sideways, or the like) over time of a feature portion (target pattern X) of the image detected by the image feature determination unit can be detected. When the operator performs an operation to follow the target pattern X, it can be considered that the operator is attempting to somehow view the feature part. Therefore, understanding the operation content by the operator on the endoscope at this time that causes the image pickup range of the image pickup apparatus 22 to change enables an interpretation of the intent of the operator. In other words, based on the position change over time of the target pattern X in the image, and information (the movement of the target and the operation by the operator) regarding the operation content to change the image pickup range by the operator performing the operation to follow the position change while viewing the position change, the observation target part to be observed by the operator can be determined from the image. The observation target part is a region including the image feature portion in the image pickup range of the image pickup apparatus, and is a target for focusing. Therefore, the focus area is decided to focus on the observation target part. Whether the operation by the operator is movement to follow the feature portion of the target in the image can be detected from the image changes, and an operation status of the operation portion may be detected using sensor information or the like and determined. It may also be expressed that the observation target part to be observed by the operator is determined from the image based on a consistency between the change over time of the feature portion of the image obtained by the image pickup unit in the image and the operation content by the operator on the endoscope to cause the image pickup range of the image pickup apparatus 22 to change.

Therefore, even if the distal end portion 21 does not move to the desired position and in the desired direction in response to the operation by the operator OP, it is possible to determine that the target pattern X is the observation target part and to focus on the target pattern X. For example, as shown in FIG. 6, even when the target pattern X does not exist at the center of the image pickup range IRa, it is possible to pick up an image with the target pattern X in focus.

In next S16, the observation target part determination unit 10 determines whether the image of the target pattern X is picked up. If the image of the target pattern X is picked up (YES in S16), the focus area determination unit 13 continues to set the region including the target pattern X to the focus area, thereby enabling the focus control unit 30 to perform focus control for the target pattern X (S17). If the image of the target pattern X is not picked up (NO in S16), the operation determination unit 12 determines whether the distal end portion 21 is moved in S18. If the operation determination unit 12 determines that the distal end portion 21 has moved (YES in S18), the operation determination unit 12 determines that observation of the target pattern X is ended and proceeds to the process to S20. In addition, if the operation determination unit 12 determines that the distal end portion 21 is not moved (NO in S18), the operation determination unit 12 determines that the target pattern X is lost (disappeared), and waits with the fixed focus, such as by remaining on standby in a target lost area, for example (S19).

The target lost area can also be described as a position in the image and a direction in the image from which the target pattern X is expected to reappear by determining in which direction, that is, the horizontal or vertical direction, the target pattern X disappeared from the image, using the center of the image obtained from the image pickup unit of the endoscope as an origin, and then waiting for the target pattern X to become visible again from that direction. The inside of the human body observed during endoscopy may perform regular repetitive motion due to pulsation or peristalsis, and parts of the human body (the lumen inner wall and the insertion portion) supporting a tube part of the endoscope may similarly move and repeat substantially the same motion, causing the same appearance to reappear. Therefore, remaining on standby, that is, focusing on a region within the image pickup range where the reappearance of the target pattern X is expected and waiting, is also effective. It may also be expressed that, at this time, if the observation target part to be observed by the operator cannot be detected in the above image, a standby region for focusing on the pickup range can be set according to a disappearance process of the above target part out of the image. To determine the position change over time of the target pattern X in the image, by sequentially recording the obtained images in the memory, these images are compared and movement of the position of the recorded feature image part is determined, and the position change over time of the target pattern X in the image can be determined. To determine the position change over time of the observation target part to be observed by the operator, an endoscope focus control device may be provided that includes a memory configured to record the obtained images in time series, and an analysis unit (one function of the image determination unit 11 in FIG. 1) configured to make a comparison considering image pickup timings of respective recorded images (for example, generating a graph with time on the horizontal axis and position in the image on the vertical axis, analyzing a pattern of the graph, determining if it is a repetitive type or a movement in a specific direction type, and the like) to analyze the direction of the position change and a position change pattern of the above observation target part in the images. In above S13, the operation determination unit 12 determines whether there are the image changes due to the movement of the organ, not due to the operation by the operator OP, but may analyze the direction and the pattern of the position change over time in the image as described here. If it can be determined whether the position change is the repetitive type or the movement in the specific direction type, in the case of the repetitive type, it may be guided to wait without further operation, and in the case of the movement in the specific direction type, it may be guided to observe in the specific direction by the image pickup unit.

In next S20, the operation determination unit 12 determines whether insertion or withdrawal of the endoscope (insertion portion 21a) is performed. If the insertion or withdrawal of the endoscope is not performed, the operation determination unit 12 returns the process to S19, and if the insertion or withdrawal of the endoscope is performed, the operation determination unit 12 returns the process to S5 of FIG. 2. If the endoscope is inserted into the lumen and the observation target part is observed, image pickup is performed such that the observation target part is automatically focused on.

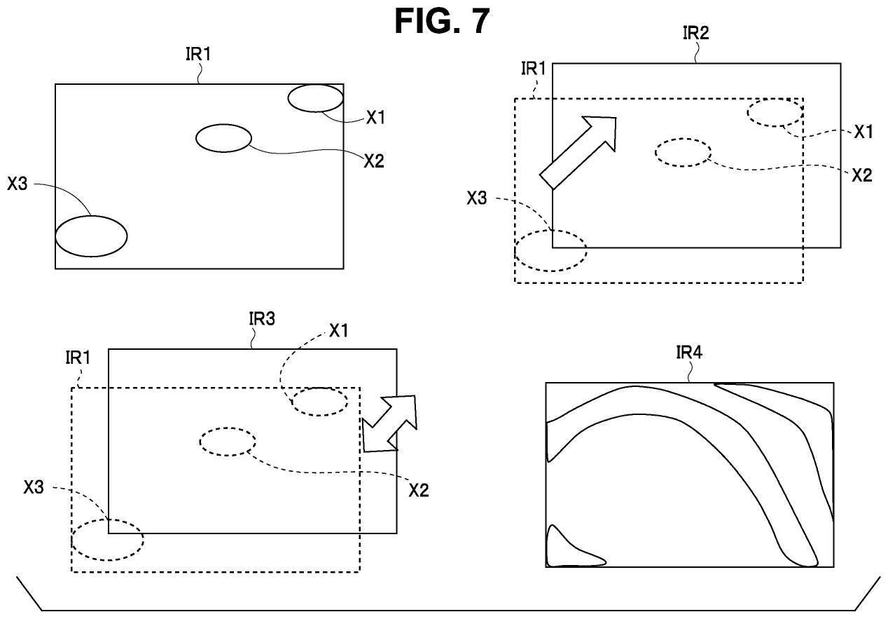

FIG. 7 is an explanatory illustration for explaining examples where a plurality of target patterns exist and where the observation target part does not include a specific pattern. FIG. 7 shows the image pickup range of the image pickup apparatus 22 by a rectangular frame, similar to FIG. 6.

The upper left section of FIG. 7 shows the example in which a plurality of target patterns X1, X2, and X3 such as lesion parts exist in an image pickup range IR1. Here, it is assumed that an operator such as a physician attempts to observe the target pattern X1, and performs an operation to move and change an orientation of the distal end portion 21, or the like, as indicated by the arrow in the upper right section of FIG. 7, thereby causing the image pickup range to change from the image pickup range IR1 to the image pickup range IR2. At this stage, the target pattern X2 is at the center of the image pickup range IR2, making it impossible to focus on the target pattern X1.

At that time, the operator such as the physician further performs the operation to move or change the orientation of the distal end portion 21. The lower left section of FIG. 7 shows a state in this case, and as indicated by the arrow in the lower left section of FIG. 7, an image pickup range IR3 is not fixed in one direction but is repeatedly adjusted or moved in small motions. In this case, the operation content determination unit 14 determines that the distal end portion 21 is not moved to the desired position and in the desired direction, despite the operator attempting to position the distal end portion 21 to the position and in the direction suitable for image pickup of the observation target part. That is, the operation content determination unit 14 determines that the operator is attempting to further move the image pickup range in the direction indicated by the arrow in the upper right section of FIG. 7, determines the target pattern X1 existing in this direction as the observation target part, and sets the region including the target pattern X1 to the focus area. Therefore, even if the target pattern X1 is not located at the center of the image pickup range, it is possible to focus on the target pattern X1.

In addition, the lower right section of FIG. 7 shows an example of an image pickup range IR4 in a case where the target pattern such as the lesion part does not exist. The lower right section of FIG. 7 shows that folds inside the lumen are included in the image pickup range, but the target pattern such as the lesion part does not exist. Even when no pattern such as the lesion part exists, the operator such as the physician may wish to observe a specific part as the observation target part. Even in such a case, in the present embodiment, based on the image changes based on the operation by the operator or the operation information based on the operation on the operation portion 25, the operation content determination unit 14 determines the operation by the operator to follow the observation target part for observation. The focus area determination unit 13 determines a region in a direction based on the operation by the operator and having a predetermined size as the focus area. Therefore, even if the target pattern does not exist, it is possible to focus on the observation target part to be observed by the operator.

Therefore, in the present embodiment, a status where the operator operates the endoscope is detected to infer an intent of the operator, and the observation target part which the operator wishes to observe is identified, and thereby control is performed to focus on the part. Thus, this facilitates image pickup reflecting the intent of the operator and reduces the time required for observation and medical examination.

The present disclosure is not limited to the above embodiment as it is, and can be embodied by modifying constituent elements within the scope not departing from the gist thereof in the implementation phase. In addition, various disclosures can be formed by combining the plurality of constituent elements disclosed in the above embodiment as appropriate. For example, some constituent elements of the all constituent elements shown in the embodiment may be deleted. Furthermore, the constituent elements among the different embodiments may be combined as appropriate.

Furthermore, among the technologies described herein, many of the control and functions mainly described in the flowcharts can be set by a program, and by having a computer read and execute such a program, the above-described control and functions can be implemented. The entire or parts of the program can be recorded or stored, as a computer program product, on a portable medium such as a flexible disk, a CD-ROM, and a non-volatile memory, or a storage medium such as a hard disk and a volatile memory, and can be distributed or provided at the time of product shipment or via a portable medium or a communication line. The user can easily implement the endoscope focus control device of the present embodiment by downloading the program via a communication network and installing it on the computer, or by installing the program from the recording medium on the computer.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another.

The term “processing circuitry,” as used herein, refers to any hardware or combination of hardware and software configured to execute the operations described. The term “processing circuitry” is a broad structural term that encompasses, without limitation, general-purpose processors (e.g., CPUs, GPUs), microcontrollers, digital signal processors (DSPs), application-specific integrated circuits (ASICs), field-programmable gate arrays (FPGAs), programmable logic devices (PLDs), and discrete logic circuits. In addition to logic or execution units, the processing circuitry may explicitly include or be integrally coupled to memory (e.g., registers, cache, RAM, ROM, or other storage media) that stores data, software, firmware, or instructions contributing to the processing operations. Accordingly, the processing circuitry may be implemented as a specialized hardware circuit having fixed logic, a programmable circuit executing instructions stored in an internal or external memory, or any combination thereof. The processing circuitry may be configured as, or include, one or more processors. Thus, the term “processor” used in the description of embodiments is to be understood as a specific example of the processing circuitry or as a component included within the processing circuitry. Furthermore, the processing circuitry may be distributed across multiple devices or locations (e.g., cloud computing) or consolidated within a single device. The term “processing circuitry” implies a concrete structure and is not intended to be construed as a purely functional “means” lacking structural support.

The term “non-transitory computer-readable (storage) medium” refers to any tangible device or medium capable of storing code or data for access by a computer or processing circuitry. This term encompasses a single storage medium or a group of multiple storage media, which may be locally or remotely distributed (e.g., across a network, in a cloud computing environment, or within a distributed ledger system) and may collectively store information in a coordinated or distributed manner. Examples of such media include, but are not limited to, non-volatile media (e.g., optical disks, magnetic disks, flash memory, ROM) and volatile media (e.g., dynamic memory, RAM, registers, buffers, and caches). Importantly, the term “non-transitory” is intended to exclude only transitory propagating signals per se (e.g., carrier waves, electromagnetic waves, or digital signals in transit through a transmission medium) and does not exclude statutory subject matter such as volatile memory where data is stored temporarily.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

In this specification and the appended claims, the singular forms “a,” “an,” and “the” include plural referents unless the context clearly dictates otherwise. For example, reference to “a recess” includes a single recess as well as a plurality of recesses.

Terms of degree, such as “substantially,” “about,” “approximately,” and “generally,” as used herein, represent a reasonable amount of deviation from the specified value or state such that the end result is not significantly changed. These terms should be construed as including a deviation of at least ±5% or ±10% of the modified value, or a deviation recognized by a person of ordinary skill in the art, unless otherwise indicated.

As used herein, geometric, spatial, or directional terms such as “parallel,” “perpendicular,” “orthogonal,” “intersecting,” “polygon,” or specific angles (e.g., “45 degrees”) are not intended to require strict mathematical or geometric exactness. Rather, these terms should be understood to encompass substantial approximations, manufacturing tolerances, and practical variations recognized by those skilled in the art, unless otherwise explicitly claimed.

In the present disclosure, terms such as “same shape,” “uniform shape,” or “common shape” refer to having the same general profile, contour, or topological structure (e.g., both being T-shaped), and do not necessarily require identical dimensions, sizes, scales, or proportions, unless explicitly stated otherwise.

Non-limiting examples according to aspects of the present disclosure will be described in the following clauses:
Clause 1: An endoscope focus control device comprising:
   an image feature determination unit configured to determine features of images obtained by an image pickup apparatus provided at a distal end portion of an endoscope, and to detect an image feature portion in the image; and
   a decision unit configured to determine an observation target part to be observed by an operator of the endoscope from the images, based on a consistency between a position change over time of the image feature portion in the image, and an operation content by the operator on the endoscope that causes an image pickup range of the image pickup apparatus to change, and decide the observation target part as a target for focusing.
Clause 2: The endoscope focus control device according to clause 1,
   wherein if the observation target part is not detected in the images, a region where a reappearance of the observation target part within the image pickup range is expected can be set as a target for focusing according to a disappearance process of the observation target part out of the image.
Clause 3: The endoscope focus control device according to clause 1, further comprising:
   a memory configured to record the images obtained from the image pickup apparatus in time series to determine the position change over time of the observation target part; and
   an analysis unit configured to compare the recorded images and analyze a direction of the position change and a position change pattern of the observation target part in the images.
Clause 4: The endoscope focus control device according to clause 3, wherein the analysis unit is, in order to analyze the direction of the position change and the position change pattern of the observation target part in the images, configured to read the images from the memory and compare the images, generate a graph with time on a horizontal axis and position in the image on a vertical axis to analyze the position change pattern on the graph, and determine whether the position change pattern of the observation target part is a repetitive type or a movement in a specific direction type.
Clause 5: A focus control method comprising:
   determining features of images obtained by an image pickup apparatus provided at a distal end portion of an endoscope, and detecting an image feature portion in the image; and
   determining an observation target part to be observed by an operator of the endoscope from the images, based on a consistency between a position change over time of an image feature portion in the image, and an operation content by the operator on the endoscope that causes an image pickup range of the image pickup apparatus to change, and deciding the observation target part as a target for focusing.
Clause 6: A non-transitory computer-readable storage medium storing a focus control program configured to, when executed by a computer, cause the computer to execute a procedure, the procedure comprising:
   determining features of images obtained by an image pickup apparatus provided at a distal end portion of an endoscope, and detecting an image feature portion in the image; and
   determining an observation target part to be observed by an operator of the endoscope from the images, based on a consistency between a position change over time of an image feature portion in the image, and an operation content by the operator on the endoscope that causes an image pickup range of the image pickup apparatus to change, and deciding the observation target part as a target for focusing.
Clause 7: An endoscope focus control device comprising:
   a determination unit configured to determine an operation content by an operator on an endoscope, the operation content causing an image pickup range of an image pickup apparatus provided at a distal end portion of the endoscope to change based on a time-series change in images sequentially obtained in time series by the image pickup apparatus; and
   a decision unit configured to determine an observation target part to be observed by the operator from the images based on a determination result of the operation content, and decide a region including the observation target part in the image pickup range of the image pickup apparatus as a focus area that is a target for focusing.
Clause 8: The endoscope focus control device according to clause 7,
   wherein the determination unit is configured to determine the operation content based on operation information on a basis of an operation on the endoscope in addition to the images obtained by the image pickup apparatus.
Clause 9: The endoscope focus control device according to clause 7, further comprising:
   a focus control unit configured to perform focus control on the focus area when the determination result of the operation content indicating a transition from a state of screening by the endoscope to a state where the observation target part is observed is obtained by the determination unit.
Clause 10: The endoscope focus control device according to clause 7, Further comprising:
   a focus control unit configured to focus on the focus area when an image of the observation target part is picked up in a state where an operation by the operator is not applied to the endoscope.
Clause 11: The endoscope focus control device according to clause 7,
   wherein the determination unit is configured to, after the time-series change in the images is determined in a state where an operation by the operator is not applied to the endoscope, identify the observation target part and determine starting of focus control for focusing on the observation target part, by determining the operation content by the operator corresponding to the time-series change, the operation content causing the image pickup range of the image pickup apparatus to change.
Clause 12: The endoscope focus control device according to clause 7,
   wherein the determination unit is configured to determine the operation content based on a time-series change in a pattern in the images obtained by the image pickup apparatus.
Clause 13: The endoscope focus control device according to clause 7,
   wherein the determination unit is configured to determine an operation by the operator to cause the image pickup range to move corresponding to movement of the observation target part relative to the image pickup range, as the operation content.
Clause 14: The endoscope focus control device according to clause 13,
   wherein the determination unit is configured to determine a bending operation by the operator on the endoscope to cause the observation target part to be positioned at a center of the image pickup range, as the operation content.
Clause 15: The endoscope focus control device according to clause 14,
   wherein the observation target part is a lesion part, and
   wherein the determination unit is configured to, when the lesion part exists in a plurality in the image pickup range, decide one of the plurality of lesion parts as the observation target part based on the bending operation.
Clause 16: a focus control method comprising:
determining an operation content by an operator on an endoscope, the operation content causing an image pickup range of an image pickup apparatus provided at a distal end portion of the endoscope to change based on a time-series change in images sequentially obtained in time series by the image pickup apparatus; and
   determining an observation target part to be observed by the operator from the images based on a determination result of the operation content, and deciding a region including the observation target part in the image pickup range of the image pickup apparatus as a focus area that is a target for focusing.
Clause 17: A non-transitory computer-readable storage medium storing a focus control program configured to, when executed by a computer, cause the computer to execute a procedure, the procedure comprising:
   determining an operation content by an operator on an endoscope, the operation content causing an image pickup range of an image pickup apparatus provided at a distal end portion of the endoscope to change based on a time-series change in images sequentially obtained in time series by the image pickup apparatus; and
   determining an observation target part to be observed by the operator from the images based on a determination result of the operation content, and deciding a region including the observation target part in the image pickup range of the image pickup apparatus as a focus area that is a target for focusing.
Clause 18: An endoscope focus control device comprising:
   an image feature determination unit configured to determine features of images obtained by an image pickup apparatus provided at a distal end portion of an endoscope, and to detect an image feature portion in the image;
   a determination unit configured to determine an operation content by an operator on the endoscope, the operation content causing an image pickup range of the image pickup apparatus to change based on a position change of the image feature portion; and
   a decision unit configured to determine an observation target part to be observed by the operator from the images based on a determination result of the operation content, and decide a region including the observation target part including the image feature portion in the image pickup range of the image pickup apparatus as a focus area that is a target for focusing.
Clause 19: A focus control method comprising: determining features of images obtained by an image pickup apparatus provided at a distal end portion of an endoscope, and detecting an image feature portion in the image;
   determining an operation content by an operator on the endoscope, the operation content causing an image pickup range of the image pickup apparatus to change based on a position change of the image feature portion in the image; and
   determining an observation target part to be observed by the operator from the images based on a determination result of the operation content, and deciding a region including the observation target part including an image feature portion in the image pickup range of the image pickup apparatus as a focus area that is a target for focusing.
Clause 20: A non-transitory computer-readable storage medium storing a focus control program configured to, when executed by a computer, cause the computer to execute a procedure, the procedure comprising:
   determining features of images obtained by an image pickup apparatus provided at a distal end portion of an endoscope, and detecting an image feature portion in the image;
   determining an operation content by an operator on the endoscope, the operation content causing an image pickup range of the image pickup apparatus to change based on a position change of the image feature portion in the image; and
   determining an observation target part to be observed by the operator from the images based on a determination result of the operation content, and deciding a region including the observation target part including the image feature portion in the image pickup range of the image pickup apparatus as a focus area that is a target for focusing.

What is claimed is:

1. A focus control device for an endoscope, comprising:
a connection interface; and
processing circuitry configured to:
receive, via the connection interface, images obtained by an imaging device disposed at a distal end portion of the endoscope;
detect a change in the images;
determine an observation target site from the images based on a correspondence between the detected change in the images and an operation to change an imaging range of the imaging device;
determine the observation target site as a focusing target; and
perform focus control of the imaging device based on the focusing target.

2. The focus control device according to claim 1,
wherein the processing circuitry is further configured to:
in detecting the change in the images, determine features of the images and detect an image feature portion in the images; and
determine the observation target site based on a consistency between a position change over time of the image feature portion in the images and the operation to change the imaging range, as the correspondence between the detected change in the images and the operation.

3. The focus control device according to claim 2,
wherein the processing circuitry is further configured to, when the observation target site is not detected in the images, set as the focusing target a region where a reappearance of the observation target site within the imaging range is predicted, based on a disappearance process of the observation target site out of the imaging range.

4. The focus control device according to claim 2,
wherein the processing circuitry comprises a memory configured to store time-series image data of the images obtained by the imaging device, and
wherein the processing circuitry is further configured to compare the images of the time-series image data stored in the memory and analyze a position change direction and a position change pattern of the observation target site in the images, thereby determining a position change over time of the observation target site.

5. The focus control device according to claim 4,
wherein the processing circuitry is further configured to:
generate a graph showing time on a horizontal axis and a position of the observation target site on a vertical axis, by comparing the images of the time-series image data read from the memory; and
analyze the position change pattern of the observation target site based on the graph, thereby determining whether the position change pattern of the observation target site is a repetitive type or a specific-direction movement type.

6. The focus control device according to claim 1,
wherein the processing circuitry is further configured to:
determine the operation to change the imaging range, based on a time-series change in the images sequentially obtained by the imaging device; and
determine the observation target site based on the determined operation, using the correspondence between the detected change in the images and the operation, and
wherein the focusing target corresponds to a focus area including the observation target site within the imaging range of the imaging device.

7. The focus control device according to claim 6,
wherein the processing circuitry is further configured to determine the operation based on operation information indicative of a manipulation applied to the endoscope, in addition to the time-series change in the images.

8. The focus control device according to claim 6,
wherein the processing circuitry is further configured to perform focus control for the focus area when the determined operation indicates a transition from a screening state of the endoscope to an observation state for the observation target site.

9. The focus control device according to claim 6,
wherein the processing circuitry is further configured to perform focus control for the focus area when the observation target site is imaged with no manipulation applied to the endoscope.

10. The focus control device according to claim 6,
wherein the processing circuitry is further configured to:
determine the time-series change in the images with no manipulation applied to the endoscope; and
determine the operation to change the imaging range of the imaging device corresponding to the determined time-series change, thereby determining the observation target site and making a determination to start focus control for the focus area.

11. The focus control device according to claim 6,
wherein determining the operation based on the time-series change in the images comprises determining the operation based on a time-series change in a pattern in the images.

12. The focus control device according to claim 6,
wherein the operation to change the imaging range includes an operation to move the imaging range in response to a movement of the observation target site relative to the imaging range.

13. The focus control device according to claim 12,
wherein the operation to change the imaging range includes a bending operation applied to the endoscope to place the observation target site at a central area of the imaging range.

14. The focus control device according to claim 13,
wherein the observation target site is a lesion, and
wherein determining the observation target site comprises determining, when a plurality of lesions exist within the imaging range, one of the plurality of lesions as the observation target site based on the bending operation.

15. The focus control device according to claim 1,
wherein the processing circuitry is further configured to:
in detecting the change in the images, determine features of the images and detect an image feature portion in the images;
determine the operation to change the imaging range, based on a position change of the image feature portion in the images; and
determine the observation target site based on the determined operation, using the correspondence between the detected change in the images and the operation, and
wherein the focusing target corresponds to a focus area that includes the observation target site including the image feature portion within the imaging range of the imaging device.

16. A focus control method implemented by processing circuitry communicably connected to an endoscope, the focus control method comprising:
receiving images obtained by an imaging device disposed at a distal end portion of the endoscope;
detecting a change in the images;
determining an observation target site from the images based on a correspondence between the detected change in the images and an operation to change an imaging range of the imaging device;
determining the observation target site as a focusing target; and
performing focus control of the imaging device based on the focusing target.

17. A non-transitory computer-readable storage medium storing instructions executable by processing circuitry communicably connected to an endoscope, the instructions being configured to, when executed by the processing circuitry, cause the processing circuitry to:
receive images obtained by an imaging device disposed at a distal end portion of the endoscope;
detect a change in the images;
determine an observation target site from the images based on a correspondence between the detected change in the images and an operation to change an imaging range of the imaging device;
determine the observation target site as a focusing target; and
perform focus control of the imaging device based on the focusing target.
